(19) United States
(12) Patent Application Publication
(10) Pub. No.: US 2008/0177217 A1
(43) Pub. Date: Jul. 24, 2008

(54) Taurolidine Formulations and Delivery: Therapeutic Treatments and Antimicrobial Protection Against Bacterial Biofilm Formation

(76) Inventor: Hans-Dietrich Polaschegg, Kostenberg (AT)

Correspondence Address: Mark J. Pandiscio;Pandiscio & Pandiscio, 470 Totten Pond Road, Waltham, MA, 02451, US

(21) Appl. No.: 11/596,525

(22) PCT Filed: May 16, 2005
(86) PCT No.: PCT/EP05/05438

§ 371 Date: Sep. 27, 2007

Related U.S. Application Data
(60) Provisional application No. 60/571,272, filed on May 14, 2004.

(51) Int. Cl. A61K 31/549; A61M 31/00; A61M 25/00; A61F 13/00; A61M 35/00; A61M 15/00; A61M 16/04; A61M 29/02; A61M 1/00; A61P 31/04

(52) U.S. Cl. 604/6.16; 514/222.5; 604/285; 604/265; 604/304; 604/295; 128/203.15; 128/207.14; 604/103.01

(57) ABSTRACT

Treating of localized bacterial infection, comprising locally applying Taurolidine to the infection. A device for insertion into the body, the device comprising Taurolidine to render the device infection resistant. A medication for treating bacterial infections, comprising Taurolidine carried by one of: gels, liquid, thixotropic gels, colloidal mixtures, dispensal suspensions, injectable polymers, or a microparticle. A method for treating blood, comprising: removing blood from the body; treating the blood with Taurolidine; and returning the treated blood.

### REFERENCE TO PENDING PRIOR PATENT APPLICATION

I claim benefit to:

(1) pending prior U.S. Provisional Patent Application Ser. No. 60/571,272, filed May 14, 2004 by Hans-Dietrich Polaschegg for TAUROLIDINE FORMULATIONS AND DELIVERY: THERAPEUTIC TREATMENTS AND ANTIMICROBIAL PROTECTION AGAINST BACTERIAL BIOFILM FORMATION (Attorney's Docket No. POLA-02 PROV);

(2) pending prior U.S. patent application Ser. No. 10/769,961, filed Feb. 2, 2004 by Hans-Dietrich Polaschegg for PREVENTION OF INDWELLING DEVICE RELATED INFECTION: COMPOSITION AND METHODS (Attorney's Docket No. POLA-01); and

(3) pending prior European Patent Application No. 03002292.5, filed Feb. 3, 2004 by Hans-Dietrich Polaschegg.

### FIELD OF THE INVENTION

This invention relates to methods of use, formulations and medical device designs using Taurolidine, and mixtures comprising Taurolidine delivered to specific local areas for sustained periods to provide: (i) therapeutic treatment of infection; and (ii) protection against attachment, and/or colonization, or eradication of colonization, on device surfaces which store or convey microbes to body fluids or contact tissue; and (iii) extracorporeal blood processing treatment to treat sepsis.

### BACKGROUND OF THE INVENTION

Taurolidine (4,4′Methlyen-bis(tetrahydro-2H-1,4-thiadiazin-1,1dioxide)), a derivative of the aminosulphonic acid taurine, is an old bactericidal agent with a broad spectrum of antimicrobial activity including Gram positive and Gram negative bacteria and fungi.^{1,2} The molecule is characterized by a unique array of attributes including low toxicity and high safety margin, neutralizing activity against endotoxins, and anti-adherence properties. Taurolidine has been tested for its antimicrobial action against most clinically significant bacteria and is an effective agent against substantially all of the most clinically significant bacteria. Furthermore, and significantly, substantially no evolution of bacterial resistance has been observed in more than 25 years of clinical trials.

The Taurolidine compound has been commercially available in several European countries since approximately the 1980's for administration to the peritoneal cavity as a lavage for treatment of peritonitis. The commercially-available compound is made by Geistlich Pharma AG of Wolhusen, Switzerland.

A second commercial application for Taurolidine was initiated in Europe in approximately 2001 for a mixture of Taurolidine and citrate, used as catheter lock solution in hemodialysis for the prevention of catheter-related infection and commercialized by Biolink Corporation of Massachusetts, USA.

Taurolidine appears to be a particularly promising antimicrobial chemical entity. Currently, many antibiotics are becoming ineffective due to bacteria developing resistance against the very antibiotics which have been used most effectively against them. This emergence of “bacterial resistance” has become a major health threat, not only in hospitals, but also in other areas. The driving force for development of resistance is the natural selection of bacteria survivors over non-survivors, following the laws of evolution. Weaker bacteria do not survive the antibiotic environment and only survivors replicate, thus becoming the dominant species. Bacteria exist in tiny niches by the hundreds of billions. Bacteria have a short reproduction time of a few hours. The convergence of short reproduction time and the vast numbers in tiny spaces greatly speeds up the manifestations of adaptation, compared to more complex life forms.

Many virulent bacteria are becoming resistant to mainline antibiotics.³ Concurrent with this threat, the pharmaceutical industry has diverted most new R&D resources away from antibiotic development, which is evidenced by a large drop in new antibiotics reaching the marketplace. Absence of new antibiotics is becoming critical as doctors face a diminished selection of effective antimicrobial agents.⁴

Taurolidine's unique properties provide a number of medically beneficial actions which are not normally provided by one drug alone. Table 1 lists some useful key attributes of Taurolidine which can be harnessed for the new treatments and prophylaxis of indwelling devices provided in accordance with the present invention.

Taurolidine was first used commercially in Europe as an antimicrobial for peritoneal lavage for peritonitis around 1980. Geistlich scientists and other researchers were quite active in performing many observational studies and generating patents for various uses beginning in the early 1970s. However, only two drugs are believed to have reached the marketplace: the Geistlich peritoneal lavage solution described previously and Biolink's Taurolidine catheter lock which was introduced around 2001. Other Taurolidine applications were not accepted into medical practice because of certain Taurolidine characteristics which inhibit clinical utility. Taurolidine has critical limitations, and these limitations need to be overcome in order to achieve successful clinical results. Taurolidine's characteristics which limit clinical utility are listed in Table 2.

Several previous clinical studies hypothesized that Taurolidine could successfully replace conventional antibiotics treatment which is not likely for systemic applications. These treatments included IV injection/infusion, open wound application of simple gels or liquid formulations, topical oral applications and inhalation therapy. However, the Taurolidine characteristics identified in Table 2 often contributed to clinical disappointments. Successful outcomes in treatment using Taurolidine are dependent on obtaining the beneficial characteristics (Table 1) while concurrently circumventing or nullifying the negative characteristics (Table 2), which together compromise Taurolidine's therapeutic potential.

Clinical trials in 1997 and 1998 were conducted to evaluate a new hemodialysis port vascular access. These trials revealed that blood stream infections were caused by bacteria from biofilm colonization of the catheter blood conduit. Subsequently, it was discovered that nearly all intravenous (IV) catheters become contaminated with a microbial biofilm, generally on the inner surface, and this is believed to be the main cause of catheter-related bloodstream infections (CRBI). Some reports describe successful use of antibiotic locks as a way to prevent biofilm contamination and resolve high CRBI. However, this is generally not an acceptable solution on a large scale because of the potential emergence of bacterial resistance.

Serendipitously, it was discovered that Taurolidine, a non-antibiotic, was a possible advancement to antibiotic use as a prophylactic agent. It seemed ideal because it had broad spectrum action and did not induce bacterial resistance^{1, 5-8}. The Taurolidine lock made a big impact in reducing infections related to the port and in catheters, and lowered hospitalization and mortality dramatically ^{9,10}. The results were considered a major advancement producing prophylaxis even against “Superbugs”, the complete absence of biocompatibility problems and no emergence of bacterial resistance.

This success occurred even though a 25 year history of many different Taurolidine applications failed. Projects to understand the underlying reasons for these failures in septic shock treatment, osteomyelitis results, and oral infections were undertaken.

This work resulted in a set of principles on optimum medical use of Taurolidine as a drug and antimicrobial agent for indwelling devices to achieve the benefits of the attributes listed in Table 1. Information from many sources revealed several medical needs which had potential improvement with correct and sufficient delivery of Taurolidine. These include topical skin infection treatments for burn healing, skin disorders (atopic dermatitis, chronic open leg sores, rosacia, etc.), various cancer treatments, breast implanting techniques, various tooth infections and healing aids. Principles which were elucidated to provide means to optimize Taurolidine's presence at a body site were key to achieving successful outcomes and the delivery variables were substantially different than antibiotics.

### SUMMARY OF THE INVENTION

### Analysis of Taurolidine Treatment Failure

Pharmacokinetics is the study of the time course of a drug and its metabolites in the body following drug administration. Effective treatment with antibiotic drugs manifests a bactericidal action by establishing and maintaining an appropriate concentration of drug for a time duration at the site of bacterial colonization sufficient to kill large quantities (i.e., orders of magnitude) of bacteria and inhibit colonization. The appropriate time-dose depends on the specific bacterial strain, the body site, the concentration of drug available, the drug toxicity and the time necessary for a biocidal effect and other factors. A recent paper shows the usefulness of PK/PD analysis to determine the quantity of drug to improve the probability of eradicating clinical infections.⁴⁰

FIG. 1 illustrates the clearance of a typical antibiotic in the blood stream. It is a typical power decay curve after drug administration. Table 3 lists important variables which determine the biocidal effect on bacteria in the blood stream. The 5 factors in the upper portion of the table define the characteristics for Vancomycin, a typical antibiotic, and the characteristics for Taurolidine. Lower in the table is the portion called Surrogate Markers. These markers are Pharmacokinetic/Pharmacodynamic (PK/PD) values calculated from the concentration profiles in the body for specifics sites and drugs to guide therapy. The derived parameters have been estimated for Vancomycin (see FIG. 1, which is average values defined in the drug label with data obtained from human trials). Taurolidine Markers have been obtained from various literature sources. The Markers are useful in predicting antimicrobial drug efficacy in infection eradication. Of course, the values of the PK/PD parameters must be for the specific bacteria causing the infection and for the specific body site.

It is important to understand (and practice accordingly) that for an antimicrobial given systemically for infection eradication for a distant site, the concentration and clearances will be greatly attenuated at the remote site.

FIG. 4 shows the time course concentration of Taurolidine in body water after delivery into the circulation. Kinetic parameters from Steinbach et al.²⁸ and infusion data from Willatts et al.²⁷ were used for the calculation. Taurolidine diffuses rapidly and body water concentration is equivalent to the plasma water concentration. Because plasma and whole blood contain additional substances, plasma concentration would be 6% less and whole blood concentration approximately 25% less. The curve shows the time values for the concentration in blood water and the mean value. The lower end range for Taurolidine's MIC is approximately 0.5 mg/mL (500 μg/mL) which is never achieved. The mean concentration in the example is only 20% of the MIC.

The reasons for this limitation are the low saturation concentration of Taurolidine in aqueous solution and the limitation of infusion rates safely allowable to a patient and Taurolidine's rapid removal from the blood by the kidney. If the concentration of Taurolidine in the infusion fluid could be increased, and/or clearance could be greatly reduced, one could also improve clinical efficacy.

Lastly, one should understand that, because of these weaknesses, and because of the attenuation of a typical drug's mode of effect passing from the central blood compartment to poorly perfused compartments, the ability of systemic drug delivery of current Taurolidine drug forms to achieve efficacious results is essentially nil.

Topical Taurolidine applications do not necessarily have the same limitations as systemic administration and some of these benefits will be described in the following section. Novel ways, however, are able to improve Taurolidines systemic performance by the following:

(i) decrease clearance in the blood stream by extending the half life; this can be achieved by several techniques used with other drugs such as Pegylation techniques and other methods which slow down the clearance of Taurolidine; and/or

(ii) increase the concentration of Taurolidine by injecting or infusing much higher concentrations of Taurolidine than currently feasible; this can be accomplished by formulating solid/semi-solid particles in the form of microspheres to achieve a concentration much greater than the 2% level which is governed by the water solubility limit; these drug particles are made to deliver drug into the surrounding tissues at an appropriate rate to sustain the effective action; particles smaller than approximately 5 μm in diameter that would pass blood capillaries could be infused directly into the blood stream where they would dissolve resulting in increased Taurolidine concentration compared to delivery in liquid form.

Another novel way to increase the concentration of Taurolidine in blood is to deliver the drug to blood which has been shunted outside the body. This process circumvents the large dilutive effects from body water and the kidney removal. A practical method for the accomplishment of this technique is described below. The technique may be especially useful for treating sepsis and other diseases which have been previously found to lack efficacy with Taurolidine systemic treatment. Furthermore, variations of this extracorporeal treatment might be to return treated blood with its high Taurolidine concentration to the local sites blood vessels.

The current problem described whereby Taurolidine does not achieve therapeutic levels in the central circulation does not exist for antibiotics (e.g., Vancomycin). Vancomycin is available in highly concentrated form and is usually diluted before application. There is no physical limitation for achieving a high systemic concentration. Also, clearance is lower because the molecular weight of Vancomycin is much larger. The risk with Vancomycin is the potential toxicity at sup therapeutic concentrations. For this reason, dosing of Vancomycin must take body volumes and kidney clearance into account. Drug companies offer modeling programs for this purpose which can also be found on the Internet. Drug dosing and frequency of injection or infusion is modeled to achieve profiles similar to the profile shown in FIG. 1, with trough concentrations several times above MIC and peak concentrations below the toxicity limit.

### Concepts and Invention

Major concepts developed for the effective application for Taurolidine are described as follows.

1. Taurolidine as a simple aqueous solution will not work as a systemic drug. The drug cannot be delivered into the bloodstream fast enough to achieve a concentration above the MIC value for almost any bacterial strains. This is because, to be stable, the concentration of the drug in the bottle is only about 1% (i.e., 10 mg/ml), which provides a low margin for success. IV drugs quickly equilibrate with the body's water which makes up most of the mass of living creatures. Also, the peak concentration established in the blood stream will quickly decay due to rapid clearance primarily by the kidney.

a. Corollary: Local infection such as tonsillitis, ear infections or skin infection will not be eradicated by conventional systemic delivery of Taurolidine.

b. Corollary: Delivery by an extracorporeal treatment technique enables a much higher concentration of Taurolidine to come into contact with blood without simultaneous infusion of large amounts of liquid into the central blood system

2. The Taurolidine catheter lock has established that Taurolidine is an effective prophylaxis. This result is achieved from the catheter geometry providing internal space for a drug depot which contacts bacteria on the inside surface of a catheter for a considerable time and the depot is isolated from the body's clearance effects to allow the concentration to stay above the Minimum Bactericidal Concentration (MBC) for a considerable time.

a. Corollary: Seek other devices with high infection rates and determine if targeting drug to a confined site of the infection, with a drug delivery pathway not exposed to the body's clearance machinery, may provide prophylaxis.

b. Corollary: Seek similar local and confined spaces in the body proximate to infected sites to create a drug depot and delivery rate and pathway without losing drug potency by the body's clearance mechanisms over a prolonged time duration.

3. Certain conditions or the indwelling device/body interface enhance Taurolidine's clinical utility such as (i) isolation from the central blood system, or (ii) protection factors which diminish Taurolidine's concentration at its intended site. These conditions may benefit from an active replenishable delivery system delivering Taurolidine to the site needed.

a. Corollary: Devices such as endotracheal tubes for breathing and urinary catheters, both of which carry high infection risk in medical care institutions, may benefit from this idea. FIG. 2 is a schematic of a patient endotracheal tube installation and FIG. 3 is a drawing defining an embodiment of this invention.

b. Corollary: The confined space between a urinary catheter and the urinary tract and in the bladder may be a beneficial target for active delivery to reduce catheter-related urinary infection.

c. Corollary: Taurolidine is unusual in that it is capable of diffusion through the skin, unlike many antibiotics. A confined space can be constructed on the outside of the skin with a barrier membrane attached to the skin. Attachment could be simply an adhesive layer on the outer periphery similar to a round bandage. Taurolidine can be delivered to the space between the membrane and the skin (wound or ulcer, for example) by appropriate connections which penetrate the membrane. The drug space may incorporate appropriate conduits to infuse and withdraw material from the space. The Taurolidine drug forms are envisaged as various types of gels, liquids, thixotropic gels, colloidal mixtures and various dispersal suspensions which can incorporate slow release properties. Many types of infusion pumps such as the small portable insulin pumps, IMed disposable type pumps, etc. can be used for active delivery or the space can be manually replenished periodically via syringe.

d. Corollary: Other confined areas formed by body contours and openings are the outer ear, nose and nasal passages, mouth, eye/eyelids, lungs, tracts made by pus adjacent to infected teeth, vagina, and anal tract, to name a few. These spaces can be further confined by designing enclosures to provide a Taurolidine depot for sufficient delivery (time, drug concentration, area of action, infusion makeup rate, etc.) of the target drug to adjacent body sites.

4. Taurolidine is a small molecule able to pass through human skin as well as through polymeric materials passively by diffusion which is driven by a concentration gradient. Experiments were conducted to determine if Taurolidine is able to diffuse through silicone rubber at a rate sufficient to achieve clinical significance. The experiment was conducted with a HD catheter-sized silicone rubber (i.e., 2 mm ID and 0.5 mm wall thickness). This was filled with water and capped off. It was immersed in a 2% Taurolidine bath. Within 3 days the inner volume equilibrated with the Taurolidine bath. The sensing instrument was a UV-Vis spectrometer.

a. Corollary: Taurolidine can be stored as a depot on the skin to treat infection sites below the skin. Certain conditions such as stage I and stage II bedsores and highly contagious antibiotic resistant Staph aureus widely reported in otherwise healthy people¹² are sites of infection in the subdermal space These conditions would benefit from topical Taurolidine delivery via passive diffusion. Alternatively, transdermal drug delivery enhancers¹³ may be incorporated into Taurolidine to achieve higher delivery. Other means available to enhance the mass transfer rate and reach deeper subcutaneous sites may benefit by use of various devices designed for transdermal drug delivery such as ultrasonically activating the skin (e.g. with a device made by Sontra Corporation), and several other available devices for driving and controlling drug delivery transdemally¹⁴.

b. Corollary: Depot creation on the outer skin layer could be by the method described in 3c above.

c. Corollary: Taurolidine can be incorporated into a depot to be placed subcutaneously or even deep within the body. The depot should be formulated to be in a form or vehicle which slows down and extends delivery duration into subcutaneous tissue. The form may be a thixotropic gel, a carrier comprising elastomeric material or polymeric plastic, micro particles or microspheres made from a variety of biocompatible and/or bioerodable materials (see below) including injectable polymeric drug forms¹⁵, etc. Several methods of slowing down the delivery from a depot are available such as pegylation techniques¹⁶, Resomer polymers¹⁷ including gels, and Epic Therapeutics type microspheres¹⁸, to name a few. The placement of the depot could be by percutaneous injection; surgical Procedure, etc.

5. Taurolidine may be formulated into various types of microparticles such as Resomer bioerodible plastics or gels, protein solids such as the Epic Therapeutics microspheres, colloidal materials, liposomes and composite silicone/hydrogel materials¹⁹. The makeup of these Taurolidine vehicles can be designed to control the delivery speed for different applications and use.

a. Corollary: Small Taurolidine particles in the range of 0.1 to 5 μm diameter may be delivered by way of dry powder inhalers or nebulizers to reach the deep recess of the lung. Certain lung diseases will benefit by this type of Taurolidine delivery, including infectious pneumonia, Cystic Fibrosis infection complications, inflammatory complications and flare-ups caused by asthma.

b. Corollary: Microspheres comprising Taurolidine are useful as a means of creating solid or semisolid depots used for wound healing. They should be placed directly in the wound or in an enclosed space over the wound to prevent infection, reduce pain, enhance wound healing and extend the duration of active drug delivery.

c. Corollary: Microsphere additives may be incorporated into polymers or elastomers to enable antimicrobial attributes in the bulk properties of these materials which make up many types of medical devices. By dispersing the microspheres throughout the material, a higher amount of drug to be available compared to certain coating techniques. The devices which will benefit are various indwelling catheters, endotracheal tubes for breathing assistance, tympanic tubes to drain middle ear fluids, female urethra plugs for urinary incontinence²⁰, intra-urethra devices (IUD) and suturing materials, to name only a few of the many medical devices which contribute to patient infection and would benefit from Taurolidine.

6. Taurolidine is an active anti-inflammatory agent and de-activates endotoxins causing fevers (i.e., pyrogenic material). Taurolidine produces pain in most tissue when in contact at concentrations of approximately 10 mg/ml or more. Wound healing of the skin and subcutaneous tissue is a process consisting of a few sequential stages which overlap to some extent. New tissue growth may be blocked by infection or an inflammatory response which prevents subsequent healing events from taking place. Recent papers report improved and rapid healing benefit from minimal anti-inflammatory response²¹. Lastly, the healing may be blocked because the process of tissue regeneration of new tissue may be blocked and needs to be activated^{22, 23}. Removal of circulating endotoxins is thought to have a beneficial effect in septic shock. Clinical trials have not shown any beneficial effects as a result of the low concentrations achievable by systemic infusion²⁴.

a. Corollary: Topical Taurolidine application can alleviate many barriers to natural healing in bed sores, other pressure sores and chronic skin ulcers and burns. Additives to Taurolidine, including non-steroid anti-inflammatory drugs (NSAID) such as salicylic acid and sodium salicylate, local pain reducing drugs, and drugs or natural substances which trigger cells adjacent to the wound to rebuild new tissue, may be useful.

b. Corollary: Early stage bed sores can be treated with Taurolidine, or Taurolidine and additives, by driving the drug through the skin and subcutaneous tissue as previously described.

c. Corollary: Blood can be treated extracorporally so as to enable high concentrations of Taurolidine to contact blood to provide deactivation of inflammatory molecules.

### Further Aspects of the Invention

In another form of the present invention, there is provided a method for treating a localized bacterial infection, comprising locally applying Taurolidine to the bacterial infection.

In another form of the present invention, there is provided a device for treating a localized bacterial infection, wherein the device carries Taurolidine therewith.

In another form of the present invention, there is provided a device for insertion into the body, the device comprising Taurolidine, whereby to render the device infection resistant.

In another form of the present invention, there is provided a method for treating a lung infection, comprising inhaling a Taurolidine-carrying aerosol.

In another form of the present invention, there is provided a method for treating a lung infection, comprising inhaling Taurolidine particles.

In another form of the present invention, there is provided a medication for treating bacterial infections, comprising Taurolidine carried by one from the group consisting of: gels, liquids, thixotropic gels, colloidal mixtures, dispersal suspensions, and injectable polymers, the medication having a Taurolidine concentration sufficiently high, and capable of being applied to a specific region for a sufficient period of time, to treat the infection.

In another form of the present invention, there is provided a medication for treating bacterial infections, comprising Taurolidine carried by a microparticle, wherein the microparticle comprises one from the group consisting of: Resomer bioerodible plastics, Resomer bioerodible gels, microspheres, protein solid microspheres, colloidal materials, liposomes and composite silicone/hydrogel materials.

In another form of the present invention, there is provided a method for using a reusable medical device, comprising positioning the reusable medical devices in a Taurolidine solution before use.

In another form of the present invention, there is provided a composition comprising Taurolidine and various pluronic formulations.

In another form of the present invention, there is provided a composition comprising Taurolidine and Hyaluronic acid (HA) and water.

In another form of the present invention, there is provided a composition comprising Taurolidine and chitin and water.

In another form of the present invention, there is provided a composition comprising Taurolidine, and chitosan or alginate, and water.

In another form of the present invention, there is provided a composition comprising Taurolidine and cyclodextrin and water.

In another form of the present invention, there is provided a composition comprising polyethylene glycol (PEG) based hydrogel system.

In another form of the present invention, there is provided a composition comprising Taurolidine, wherein the composition is injectable and comprises a scaffold upon subcutaneous administration so as to provide a filler for a body cavity or act as a compliant implant and release the Taurolidine in vivo.

In another form of the present invention, there is provided an endotracheal tube comprising:

a tube;

an inflatable collar attached to the outer wall of the tube, the inflatable collar comprising a material permeable to Taurolidine; and

a passageway extending along the tube and connected to the inflatable collar;

whereby Taurolidine may be introduced to the inflatable collar from outside the body in a flow and pressure controlled manner so as to inflate the collar and deliver Taurolidine around the periphery of the collar and/or the tube outer surface distal to the collar.

In another form of the present invention, there is provided a method for ventilating a patient, comprising:

providing an endotracheal tube comprising:
a tube;
an inflatable collar attached to the outer wall of the tube, the outer inflatable collar comprising a material permeable to Taurolidine; and
a passageway extending along said tube and connected to the inflatable collar;

positioning the tube in the trachea;

introducing Taurolidine into the inflatable collar from outside the body in a flow and pressure controlled manner so as to inflate the collar and deliver Taurolidine around the periphery of the collar and/or the tube outer surface distal to the collar; and

ventilating the patient

In another form of the present invention, there is provided a urinary catheter comprising:

a tube;

an inflatable collar attached to the outer wall of the tube, the outer inflatable collar comprising a material permeable to Taurolidine; and

a passageway extending along said tube and connected to the inflatable collar;

whereby Taurolidine may be introduced to the inflatable collar from outside the body in a flow and pressure controlled manner so as to inflate the collar and deliver Taurolidine around the periphery of the collar and/or the catheter outer surface distal to the collar.

In another form of the present invention, there is provided a method for treating a patient, comprising:

providing a urinary catheter comprising:
a tube;
an inflatable collar attached to the outer wall of the tube, the outer inflatable collar comprising a material permeable to Taurolidine; and
a passageway extending along said tube and connected to the inflatable collar;
whereby Taurolidine may be introduced to the inflatable collar so as to inflate the collar and deploy Taurolidine around the periphery of the collar;

positioning the tube in the urethra;

introducing Taurolidine into the inflatable collar from outside the body in a flow and pressure controlled manner so as to inflate the collar and deliver Taurolidine around the periphery of the collar and/or the catheter outer surface distal to the collar.

In another form of the present invention, there is provided a Taurolidine depot adapted to be deposited within the body during a medical procedure, and thereafter deliver Taurolidine to the tissue adjacent to the body.

In another form of the present invention, there is provided a method for treating a patient, comprising:

providing a Taurolidine depot adapted to be deposited within the body during a medical procedure, and thereafter deliver Taurolidine to the tissue adjacent to the body; and

depositing the Taurolidine depot within the body during a medical procedure.

In another form of the present invention, there is provided a method for treating blood, comprising:

removing blood from the body;

treating the removed blood with Taurolidine; and

returning the treated blood to the body.

In another form of the present invention, there is provided an apparatus for treating blood, comprising:

removal apparatus for removing blood from the body;

treatment apparatus for treating the removed blood with Taurolidine; and

return apparatus for returning the treated blood to the body.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts and further wherein:

FIG. 1 shows average IV Vancomycin clearance in patients and an MIC value for commonly found bacteria;

FIG. 2 shows a schematic view of endotracheal tube placement;

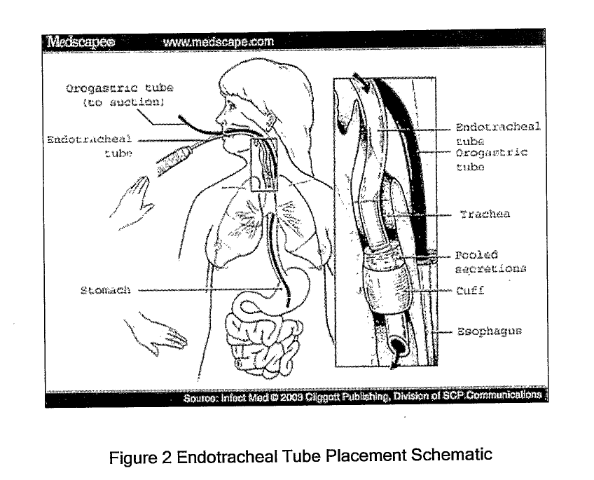

FIG. 3 shows a schematic view of advanced endotracheal tube with replenishable antimicrobial prophylaxis;

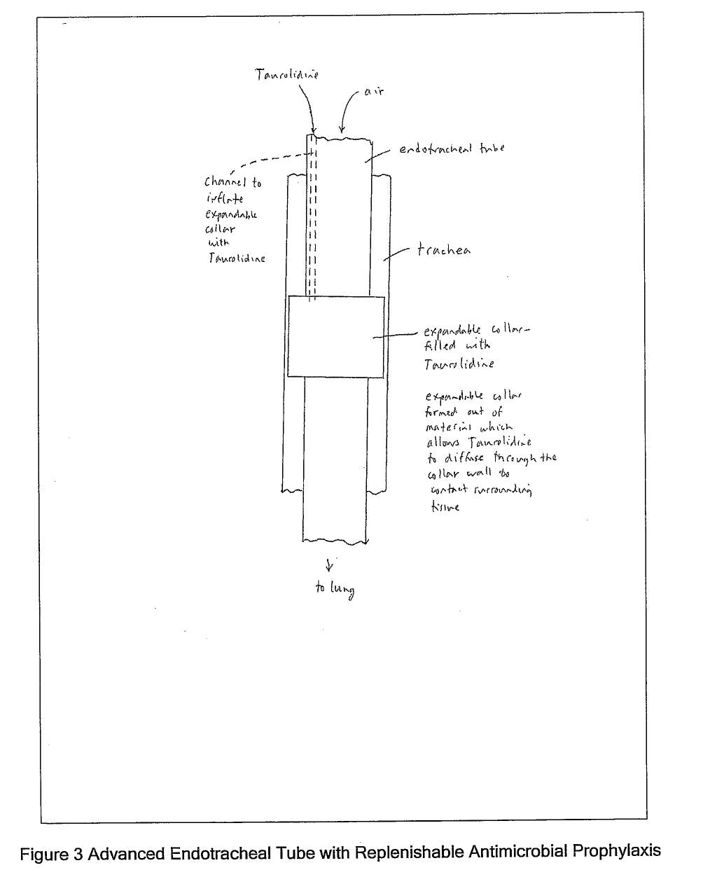

FIG. 4 illustrates drug IV concentration over 24 hours after 6 evenly spaced 2 hour infusions of 2.5 grams of Taurolidine;

FIG. 5 illustrates a standalone Taurolidine extracorporeal sepsis treatment system;

FIG. 6 illustrates a standalone Taurolidine extracorporeal sepsis treatment system with ultra-filtration fluid removal;

FIG. 7 illustrates another Taurolidine extracorpeal sepsis treatment system;

FIG. 8 illustrates batch type dialysis machine with taurolidine delivery; and

FIG. 9 illustrates a Taurolidine extracorporeal blood treatment system.

### PREFERRED EMBODIMENTS OF THE PRESENT INVENTION

### Biomedical Antimicrobial Materials—Novel Antimicrobial Elastomers for Indwelling Medical Devices

A novel antimicrobial elastomeric material may be formed with conventional biocompatible elastomers (particularly silicone rubber) by dispersing Taurolidine deposits throughout the material in a diffusible form to enable water to diffuse freely in and out of the material and the Taurolidine to diffuse out. The process preferably adds Taurolidine in solid powder form during the early stages of manufacture before final chemical curing of the material. The Taurolidine powder is mixed into the uncured ingredients in a way that uniformly disperses the Taurolidine in the bulk material (and with other additive ingredients if desired as previously described). The material can be processed in a conventional manner through the curing stage. During the processing after adding Taurolidine, the temperature should not exceed 110° C. The selection of the base elastomers is governed by the usual criteria that satisfies biocompatibility and meets the mechanical properties required for the device specifications. The quantity and form of the Taurolidine additive shall be determined by tests to achieve a delivery rate and duration of action for the design operating life. In use in the body, water may permeate the elastomer and Taurolidine particles are able to dissolve into this water and diffuse out to the surface, creating a hostile surface for bacterial attachment and colonization. The diffusion rate is based on several variables including the form of the Taurolidine and the amount, the concentration gradient, the elastomer's characteristics and the geometry of the item. Examples of migration of antimicrobial agents migrating from a polymer material are silver ions, drugs contained in silicone rubber hydrogel composites and various antibiotics¹⁸⁻²⁰.

Silicone rubber is a particularly suitable matrix for high diffusion rates for water, Taurolidine and other additives with good biocompatibility and mechanical durability in vivo.

Taurolidine and its additives can be formed into various configurations:

Dry Solid Particles—Taurolidine is usually manufactured as a dry white powder. This powder can be conventionally made into powders of a selected size or can be selected through sieving to a particular size. For purposes of making device polymers the particle size should generally not exceed 25 μm.

Coated particles—Taurolidine particles may be coated to improve the compatibility with the base polymer and can be used to calibrate the delivery characteristics. PVP (Polyvinyl Pyrrolidone) and PEG (Polyethylene Glycol) are among the possible coatings.

Solid or semi-solid microspheres—Useful microsphere forms include (i)Resomer erodeable solids and gels, (ii) Epic Therapeutics type microspheres, (iii) hydrogel microspheres, and (iv) liposome particles.

### Medical Device Examples

Several different medical device applications can benefit using construction materials comprising polymers with incorporation of Taurolidine along with other enhancing additives. The benefits include protection against bacterial colonization on the outer surfaces which increases the risks of infection, reduced inflammatory effects from implantation of a foreign object, improved biocompatibility, enhanced healing after implantation, and reduced attachment of proteins, thrombosis and other types of cells which normally attach to foreign materials. It is envisaged that the following partial list of devices would benefit from the afore-mentioned materials: urinary catheters, female urethra plugs for urinary incontinence²⁴, blood catheters, intra-spinal catheters, enteral feeding tubes, intra-ventricular shunts, drainage tubes, endotracheal tubes, active wound healing dressings, suture materials, adhesives, tympanic tubes for ear drainage, implantable ports and pumps, pacemakers, breast prosthesis, intra-uterine devices (IUD), endoscopes and other indwelling devices. Many of these devices are used for short periods and suffer from biofilm formation on the surfaces which raise the risk of causing patient infection.

Reusable medical devices (e.g., endoscopes) can be kept sterile in a Taurolidine solution. This is especially useful when the device is used in wet form.

### Medical Device Treatment Examples

Many medical treatments are possible and/or enhanced by using depot drug delivery techniques comprising Taurolidine and/or Taurolidine mixtures in a polymeric vehicle or carrier or other formulation of Taurolidine. The depot may be a carrier, may be produced in different shapes to suit the purpose such as rods, spheres, films, discs and more complex geometries, and may be placed in a wound or body cavity. The shapes can be formed to pass through tissue to facilitate removal after a dosing period. Taurolidine depots may be placed in or near the site intended for prophylaxis or treatment of infection, including sternum surgical incision sites (i.e., placement after open heart surgery before final surgical skin closure), infected bone treatment (i.e., osteomyelitis or prophylaxis), vaginal inserts for prophylaxis, small solid rods or injectable solids or thixotropic gels for insertion into the pus drainage tracks around infected teeth (periodontal disease), semi-solids or thixotropic gels and gel-gauze combination devices for placement in the void from extracted teeth.

### Bed Sores and Other Pressure Ulcer Type Chronic Ulcer

These types of chronic and often difficult to treat open wounds will benefit by use of Taurolidine formulations including combinations of the following: Taurolidine only, Taurolidine and non-steroid anti-inflammatory drug (NSAID) such as salicylic acid and sodium salicylate²⁵, Taurolidine and a local pain killer, and/or Taurolidine with a cell repair activator^{22,23}.

For open sores, the treatment method envisaged is to make a confined space over the wound with a barrier type bandage as described previously in paragraph 3c. The bandage type barrier will incorporate simple connector passages with means to seal and provide a means of delivering and withdrawing fluids from the space. Start therapy by introducing a sufficient quantity of Taurolidine formulation into the space created to enable Taurolidine to be in contact with the surface of the wound. Provide proximal contact with the open wound with complete filling of medicate. The membrane serves to protect the open ulcer as well as provide a storage space for the drug entity and facilitate delivery. The Taurolidine/bandage provides a means for eradication of bacteria and fungi and facilitating of healing, protection, pain relief and enhanced tissue growth. The bandage barrier should be replaced every few days. It should be noted that this alone will not guarantee a complete success. Permanent healing also requires a good blood supply to carry oxygen and nutrients to, and remove waste from, the tissue in the site of the ulcer. Without an adequate blood supply, healing will not reach the final stage or will relapse back to pre-treatment conditions. A vascular surgeon may be helpful in re-constructing failed blood vessels and in moving vessels to a needed place.

Early stage bedsores (e.g., before skin maceration or sore breakthrough) will best be treated by the methods defined previously. Early stage bedsores are visible by the appearance of redness, swelling, warmness of the tissue and pain. This early stage bedsore may benefit with essentially the same approach above which relies on passive diffusion for the driving mechanism. However, enhancing the delivery rate may be more appropriate if the depth and size, etc., require more drug than is available through passive diffusion. There are many ways to provide higher delivery, as noted previously. Also, a more direct way is to inject the depot into subcutaneous tissue. One form of a depot comprises various bioerodible microparticles or a gel which flows at room temperature and transforms to a solid at body temperature¹⁴.

### Inhalation Therapy

The inhalation therapy of the present invention is designed to provide a non-systemic local therapy to treat lung infections and/or inflammatory events by delivery of Taurolidine into the airways of the lungs. Specifically, the treatment is be designed to provide that sufficient amounts of Taurolidine be dispersed throughout the bronchioles and alveoli. Local directed delivery of Taurolidine will benefit many lung diseases such as pneumonia, bronchitis, asthma, infections related to cystic fibrosis and other infectious and inflammatory conditions.

In vitro microbiology tests show positive response for Taurolidine against resistant Burkholderia cepacia from adult cystic fibrosis patients. Subsequently a clinical trial was performed delivering Taurolidine to the lung via a nebulizer The clinical results indicate failure to achieve a significant reduction in bacterial load in most patients²⁶. My analysis of the study indicated that the amount of drug delivered to the lung was not sufficient to achieve a bactericidal effect. Also, the particle size of the nebulizer was not controlled to a small enough size to ensure delivery to the far reaches of the lung alveolar duct.

A particularly good carrier method for Taurolidine for inhalation is to make the particles similar to the protein microspheres developed by Epic Therapeutics which has developed clinically effective inhaled insulin for diabetes care. This method produces small microspheres approximately 1-10 um in diameter with a tight size distribution. These particles are made using safe chemicals and comprise high loadings of the drug (approximately 95%) and with low levels of safe excipients. The particle size and weight provide good aerodynamic lift properties and test results show deep penetration of the airways. The drug is delivered through a dry inhalation technique. Studies show these type particles have the appropriate aerodynamic properties to reach deep within the airways of the lung and can be tailored to achieve an appropriate capture time in the lung to be clinically effective. Other forms of Taurolidine microparticles with small particle size (less than 3 um) are also acceptable. The therapy consists of treatment 1 to 5 times per day of drug inhalation for a few weeks until the disease situation is improved. This treatment may also be applied while the patient may be simultaneously receiving conventional systemic antibiotic therapy for serious life threatening lung infections. An alternative method is to formulate the active agent Taurolidine with another active agent such as salicylic acid or sodium salicylate into microspheres or other NSAID.

### Periodontal Disease Treatment

A recent dental study was undertaken to quantify the amount of different bacteria living in the mouth. More than 150 bacterial strains were isolated and identified, some for the very first time. The large number of microbes present able to induce infection makes it difficult to treat with antibiotics. Taurolidine is ideally appropriate for its broad spectrum antimicrobial effect.

Periodontal disease is usually recognized in the form of a biofilm in the proximate location of the tooth root. It affects approximately 1.5 million people in the U.S. and many of them are treated for several months or years attempting to eradicate the infection. Some treatments use local antibiotics delivered directly into the tract along a tooth which drains pus. Bleach and other antimicrobials are also used. Several available forms of antimicrobials have been introduced into dentistry including (i) small bioerodible rods comprising active agent in Resomer material, and (ii) injectable liquids which harden into a solid at room temperature. These are placed by dentists on a periodic basis. Many of these methods suffer by not being curative, and many of them produce bad tastes and discolor the teeth to which patients object. The particular characteristics making Taurolidine superior to current techniques are (i) Taurolidine passes though the mucous lining in the mouth extremely well, (ii) it is a very broad spectrum antibiotic, (iii) it is anti-inflammatory, (iv) it is tasteless, (v) it does not discolor teeth, (vi) it is very safe, even in the cases of an accidental massive overdose, and (vii) it does not induce bacterial resistance. Furthermore, the occurrence of pain associated with Taurolidine in many tissue sites is absent in the mouth. Also, the technique described below is the preferred delivery method which can be done by the patient on a daily basis at home.

An effective treatment requires sufficient concentration of the Taurolidine for a prolonged time period (i.e., >4 hours/day for several days). The drug form must be much higher than the max aqueous vehicle, which is approximately 1%, so that even when it is dispersed within the gum tissue and is greatly diluted, the drug still has a concentration at the local tissue site that is several times higher than the MIC and exceeds the Surrogate Markers defined in Table 3. Also, the depot must contain enough drug to maintain delivery higher than the removal rate into a surrounding tissue and blood stream. The application should be such that the drug depot is substantive and not be washed away or diluted by saliva nor should it be carried away or dislodged by the tongue mechanical action.

A preferred method of treatment consists of bringing Taurolidine into contact with the infected tissue around the roots of the teeth in the gums. This can be accomplished by employing conformal trays fitting upper teeth and lower teeth as appropriate. Trays similar to this are used for holding whitening solutions for teeth treatment. These trays are intended to cover only the exposed teeth while a tray for my invention must be deeper to bring the solution into contact with the gums. Equipment for making trays for whitening teeth is supplied to dentists by commercial dental equipment manufactures. The dentist uses the equipment to make conformal trays for his tooth whitening patients.

The tray making consists of the following steps:

A. Dentist casts a female cavity form of patient's upper teeth and lower teeth. The mold material is a fast drying plaster or epoxy type material and only has to stay in the patient's mouth for a few minutes.

B. The cured mold is placed in a small tool which positions the mold on a platform and a plastic material approximately 1.5 mm thick and the 15 cm×15 cm sheet is place above the mold and the plastic is heated with lamps to soften. A vacuum is applied to the mold side and this pulls the softened plastic over the mold so that it matches the shape of the upper or lower tooth array including the aum region. In the plastic industry this is called vacuum molding. The dentist removes the tray and cuts and adjusts the tray to fit the patient's mouth. This simple manufacturing process can be adapted and modified by one skilled in the art to be optimum for making trays to carry liquid or other flowable substances such as Taurolidine (or even other antimicrobial substance or even antimicrobial agents to prevent tooth decay).

The preferred method of treating the patient's periodontal disease infection is to partially fill the tray with Taurolidine in the form of a thixotropic gel or high viscosity vehicle. Place the tray over the upper and/or lower teeth which contain the infected teeth area and fill with the drug. Maintain overnight while sleeping or some other comparable length of time. The tray can be modified by one skilled in the art to only encompass a few teeth rather than all of the upper or lower teeth with ease. Even if a major leak of Taurolidine occurs, no harm will come to the patient. People schooled in the art can easily make changes to the tray design to provide drug delivery to only the teeth to be treated.

This technique can also be adapted for treatment of infected implanted teeth and their neighbors. An infected implanted tooth has a devastating impact. Without effective treatment, the implanted tooth would be lost and possibly affect other adjacent teeth. Other gum infectious disease can be treated by this local topical treatment using Taurolidine.

An alternate technique for applying high concentration Taurolidine is to prepare an ointment which is poorly soluble in saliva, and can adhere to the exposed portion of a tooth similar to an adhesive, and Taurolidine can leave the material by passive diffusion and penetrate gum tissue.

One other example of treatment for oral infections is to apply Taurolidine to the open wound created by the act of tooth extraction. Topical delivery of Taurolidine after extraction to the open tissue prevents infection and reduces the inflammatory response. The result is faster healing, reduced swelling, less pain and reduced local hotness at the site. Taurolidine in the form of a thixotropic gel with relatively high loading of Taurolidine is the preferred formulation. It can be injected into the space and then covered with a wad of cotton or a rubber plug held in place by adjacent teeth. Adding a carrier to reduce the dissolution with saliva is a preferred approach and can be accomplished by increasing polymer content of a cross linked hydrogel vehicle.

### Topical Treatment for Middle Ear Infections

Four million children in the U.S. suffer from ear infections and are treated by antibiotics. U.S. health officials have recently begun a campaign aimed at reducing these numbers by 75% in an attempt to stem the rise in bacteria resistance. A middle ear infection is usually caused by a local colonization of bacteria in the space on inner surface of the tympanic membrane. The tympanic membrane is a thin permeable material allowing a Taurolidine pathway through it. Many children with bacterial inner ear infections would be helped by delivery of Taurolidine topically to the outer surface of the tympanic membrane. This could be accomplished by delivery of a small quantity of Taurolidine solution using a tiny tube which can be inserted down the outer ear canal and injection from a syringe. The Taurolidine can be held in place with a cotton ball. A preferred method is to use a thixotropic gel form of Taurolidine and deliver it to the space proximate to the outer surface of the Tympanic membrane.

### Improved Biocompatible Breast Prosthesis

Breast implants are a common cosmetic procedure performed in the U.S. Many women receiving the procedure are subject to a complication wherein a tissue capsule grows around the implanted prosthesis and contracts around the implant, causing a hardening effect which is painful. This capsule encapsulation is the body's normal response to implanted synthetic material, wherein the capsule isolates the foreign body from the living host. Highly biocompatible implant materials form a thinner sheath or capsule around the device. Some surgeons have observed that Taurolidine soaking of certain synthetic materials improves the biocompatibility and healing after implantation. This invention improves over current breast prosthesis. It comprises a thin permeable flexible bag containing Taurolidine gel or liquid such as saline and Taurolidine. The construction is configured so that Taurolidine will slowly diffuse outward through the bag membrane material. This provides better healing, less inflammation and less pain. Medical observations indicate a more natural feeling breast to the patient and a reduction in the capsule surrounding the implant for an extended period after the placement compared to non-Taurolidine treatment.

### Soft Eye Contacts with Improved Prophylaxis

In this form of the invention, the approach is to provide a means to load the gel eye contact material with Taurolidine daily to prevent infection. The gel material will be capable of storing an aqueous solution of Taurolidine and can be replenished daily by soaking for several hours in a high concentration Taurolidine aqueous solution.

### Orthopedic Prosthesis

In this form of the invention, the approach is to design device/drug combination products incorporating an active delivery system for Taurolidine which is available subsequently to treat a device-related infection in the event the device becomes the nidus for infection. The design envisions a small port/reservoir assembly within the orthopedic prosthesis which can be accessed via percutanious needle to administer Taurolidine as a prophylaxis immediately after the surgery or at selected future time to treat an infection.

### Bone Cements for Attachment of Orthopedic Prosthesis

In this form of the invention, the approach is to incorporate Taurolidine mixtures into bone cement which helps healing, reduces inflammation and infection.

### Ophthalmic Fluids

In this form of the invention, the approach is to incorporate Taurolidine mixtures into ophthalmic fluids to help healing, and reduce inflammation and prevents infection safely.

### Systemic Application of Taurolidine

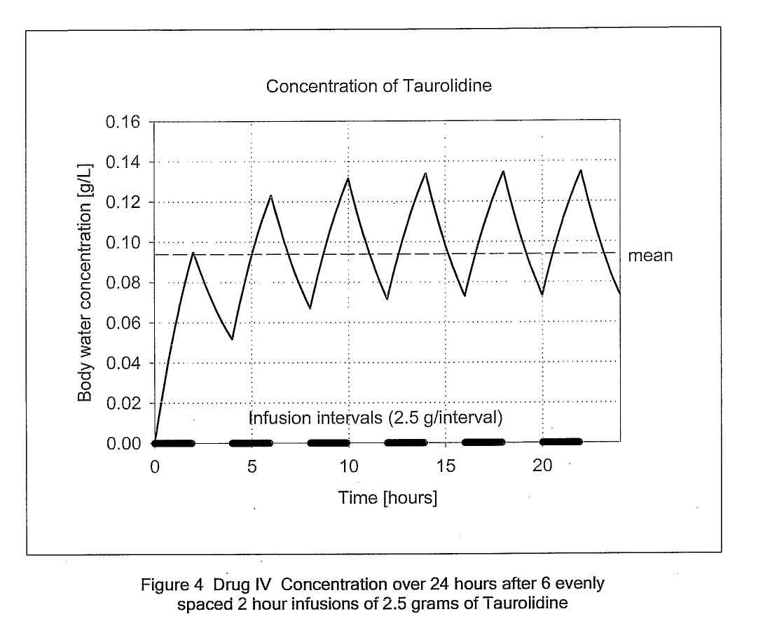

Taurolidine has anti-endotoxic properties and has been used by Willatts, et al. in the treatment of sepsis syndrome³⁷. No benefits of Taurolidine infusion was found in this study. Taking into account that Taurolidine must be present in concentrations of greater than 0.0005 (0.05%) to show minimum effectiveness, the result is no surprise. In this study, 5 g per 4 hours=30 g/day of Taurolidine were infused. When infused into blood, Taurolidine will distribute in blood water. Because of its low molecular weight, it will rapidly distribute in total body water. Because the 5 g of Taurolidine are infused over a period of two hours, distribution in the body water of approximately 40 liters for a patient with approximately 60 kg body weight (Guyton A C, Textbook of Medical Physiology, Eight Edition, W.B. Saunders Company) must be assumed. The resulting concentration is less than 0.01% after prolonged infusion (FIG. 4).

Higher concentrations of Taurolidine in blood and body can be accomplished by utilizing extracorporeal administration of Taurolidine. In this discussion, Extracorporeal treatment (ET) will be used as general term for hemodialysis (HD), hemofiltration (HF), hemodiafiltration (HDF), plasmafiltration (PF), plasmapheresis (PP), hemoperfusion (HP) and combinations of these modalities, e.g., plasma adsorption. For the following discussion the term Taurolidine means the substance and its dissociation products.

Application of extracorporeal blood treatment for the treatment of the sepsis syndrome has been previously proposed and studied based on different concepts than I claim for achieving utility³³⁻³⁶.

The molecular weight of Taurolidine is small, which means that it diffuses rapidly, resulting in a high transfer rate in the dialyzer A simple estimate shows that large quantities of Taurolidine can be transferred to the body by dialysis. At a blood flow rate of 200 mL/min using an effective dialyzer, e.g., the F50 dialyzer of Fresenius, an aqueous clearance of approximately 180 mL/min can be achieved. Multiplying with the plasma water fraction of approximately 0.55 results in a net clearance of approximately 100 mL/min. The Taurolidine concentration of dialysate is 2% (2 g/100 mL), and the dialysate flow is assumed to allow the assumed clearance (dialysate flow QD>200 mL/min). The transfer rate is then: 2 g per min (transfer rate=concentration*clearance: 2 g/100 mL*100 mL=29). This means that almost 3 kg of Taurolidine can be transferred per day, as compared to 30 g infused in the clinical trial reported by Willatts et. al.

The Willatts paper has not shown any beneficial effects of Taurolidine, but it also has not shown any statistically significant decremental effect. The safety of Taurolidine infusion cannot be extrapolated from 30 g/day to the hundredfold amount of 3000 g/day without performing appropriate tests. Mediators of sepsis are thought to be transported by the blood stream, therefore efficient treatment of whole blood or plasma by ET with Taurolidine should be sufficient, and the treatment can than be combined with a less aggressive systemic Taurolidine treatment.

For blood treatment only (eventually combined with less aggressive systemic treatment), blood can be exposed to Taurolidine by any of the available ET methods. After Taurolidine is transferred to the bloodstream in the extracorporeal circuit, blood can be circulated or stored to increase the exposure time. Before it is re-infused into the body blood (or plasma), it is dialyzed again in order to remove all or part of the Taurolidine. Alternatively, Taurolidine could also be removed by adsorption or chemical binding.

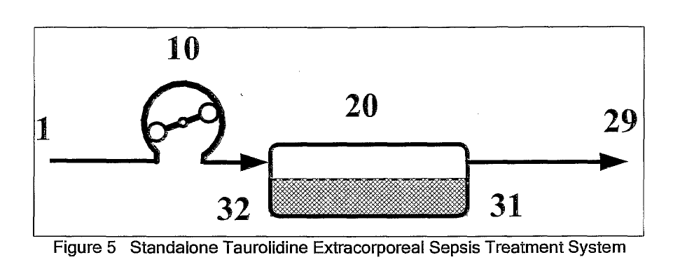

Several embodiments are described by FIGS. 5-9.

FIG. 5 shows a standalone Taurolidine ET system. Element 1 is the blood withdrawal blood connection, 10 is a blood pump, 20 is a dialyzer which is separated into a blood side part and a dialysate side part by a semi-permeable membrane. The dialysate part is filled with Taurolidine powder or slurry. The dialysate part has two connectors 31, 32 which are used for filling or replenishing the dialysate part with Taurolidine. Element 29 is the blood return connection.

For operation, the ET system is filled with a physiological fluid (saline), connected to the blood access and the pump is operated. Taurolidine must be added from time to time to the dialysate part when it is used up. This can be done in predetermined intervals, depending on the dialysate part filling volume of the dialyzer and the blood flow. Alternatively, sensors (optical) can be mounted at the dialyzer housing for detecting the exhaustion of Taurolidine. Not shown are protective systems commonly used in extracorporeal circuits like pressure monitors and air detectors.

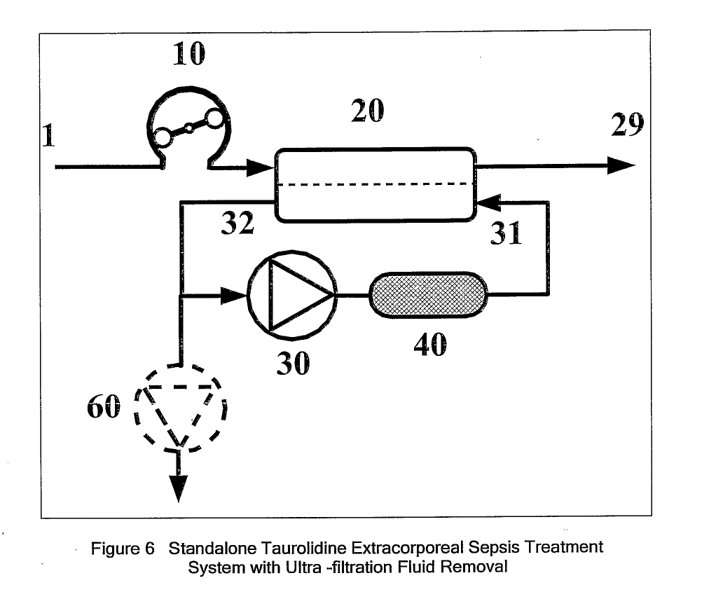

FIG. 6 shows a standalone Taurolidine ET system combined with fluid removal by ultrafiltration. The blood circuit is identical to the one described by FIG. 5. The dialysate side consists of the circulation pump 30 and a Taurolidine cartridge 40. Cartridge 40 can be closed by sieves or filter membranes to avoid escape of Taurolidine during connection to the system and of solid Taurolidine particles during operation. A sensor may be added to the circuit downstream of cartridge 40 to detect the exhaustion of Taurolidine. This sensor can measure optical parameters or density. optionally, an ultrafiltration pump 60 removes fluid from the dialysate circuit which is replaced by ultrafiltrate from blood.

For operation, the blood side and the dialysate side of the system are filled with saline. The blood side is connected to the blood access and the blood pump is operated.

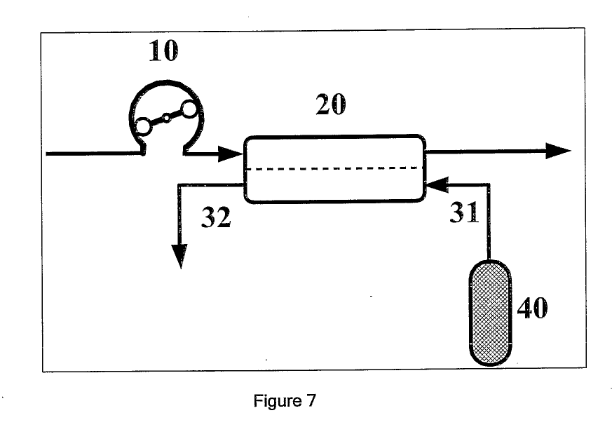

FIG. 7 shows the foregoing combinations with a conventional hemodialysis machine (not shown). The blood circuit is identical to the previous FIGS. 5 and 6. Fresh dialysate from the dialysis machine is pumped through cartridge 40 into the dialysate side of the dialyzer 20. The outlet (32) of the dialyzer is connected to the used dialysate part of the dialysis machine.

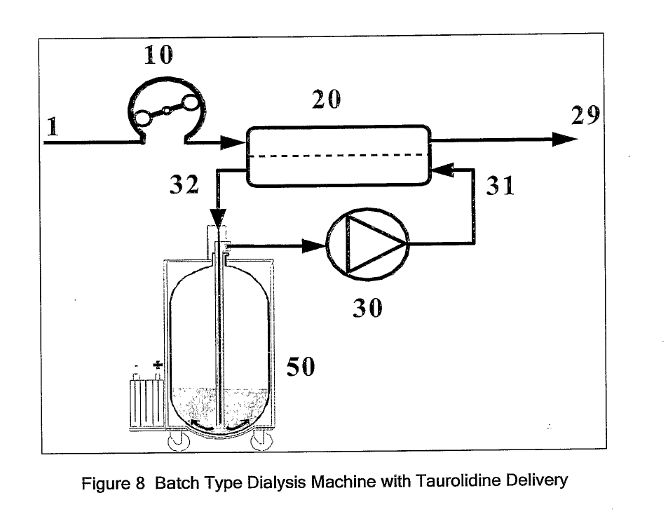

FIG. 8 shows treatment with a conventional batch-type dialysis machine (e.g., the GENIUS from FMC Germany)³⁸. This machine (50) is filled with dTaurolidine containing dialysate before the treatment. For dialysis, fresh dialysate is taken from the top of the tank, pumped by pump 30 through the dialyzer 20, and is returned to the bottom of the tank.

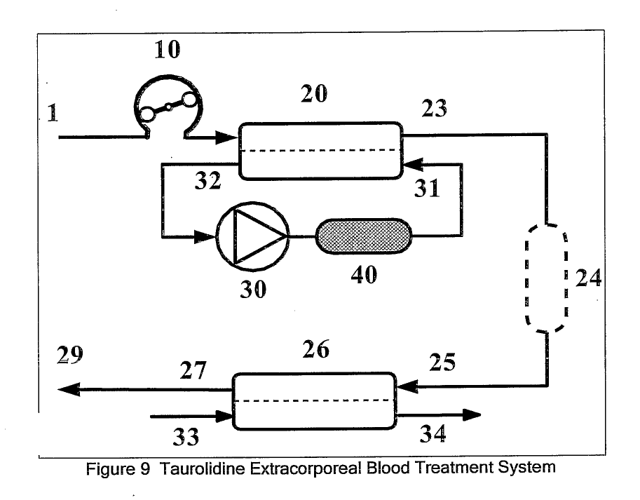

FIG. 9 shows a system for blood treatment only. As described in FIG. 6, blood is exposed to Taurolidine in dialyzer 20. From there it flows to a second dialyzer 26, where Taurolidine is dialyzed out with the help of a conventional dialysate circuit. optionally, a vessel 24 is inserted in the blood circuit between the two dialyzers to increase the exposure time.

This principle can also be applied in a system like the MARS³⁹. In this case, the Taurolidine cartridge would be placed in the dialysate circuit between the albumin coated filter/dialyzer and the secondary dialyzer.

As mentioned above, plasma can be treated instead of whole blood. In this case, any of the embodiments described above could be connected to a source of blood plasma (e.g., the plasma outlet of a plasma filter or blood centrifuge) and returned to the blood stream.

A single-access (single-needle) system can be used instead of the conventional double-access system shown by FIGS. 5-9. Such systems are typically considered to be state of the art.

### Additional Taurolidine Drug Forms

A. Injectable formulations and/or thixotropic forms, including those that change from a liquid at room temperature to a semi-solid or solid at body temperature, comprising:

Taurolidine+pluronic gel such as F127+water; or

Taurolidine+Hyaluronic acid (HA)+water (this formulation would impart antimicrobial, anti-inflammatory and cell migration enhancement properties to a drug product); or

Taurolidine+chitin (or chitosan or alginate)+water; or

Combinations of the above ingredients with surfactants added.

B. Taurolidine in the form of microspheres forming strips for bandage applications and the film can release the active ingredients. The film can be made in several layers to tailor the release rate.

C. Taurolidine+cyclodextrin+water. Formulations with solubility enhancers such as Hysolv or Resolv can be produced. The solubility enhancers can be combined with other formulations of Taurolidine

D. A polyethylene glycol (PEG) based hydrogel system could be injectable and comprise a scaffold upon subcutaneous administration to provide a filler for a body cavity or act as a compliant implant and release the Taurolidine drug in vivo.

E. Microbicides in combination with barrier contraceptives could be formulated into an aerosol, cream or foam for prophylaxis in vaginal applications.

The above formulations could be used for a variety of treatment applications, including:

Chronic wounds such as pressure ulcers, burns, surgical sites and other types of wounds

Osteotitis or osteomyelitis

Gastric ulcers from H Pylori

Wound dressings treating or prophylaxis

### Further Embodiments

It is to be understood that the present invention is by no means limited to the particular constructions herein disclosed and/or shown in the drawings, but also comprises any modifications or equivalents within the scope of the invention.

### PUBLICATION REFERENCES

1. Blenkharn, J I. Antibacterial and Related Properties of Taurolin®. Geistlich Pharma AG, Wolhusen, Switzerland. December 1998. p 1-41

2. Polaschegg H D. Taurolidine, anew antimicrobial catheter lock solution. Dialysis Times 2000: 7: 1-8

3. Zuger, A. Bacteria Run Wild, Defying Antibiotics. New York Times Newspaper, New York, N.Y., USA. Mar. 2, 2004

4. Rosenthal M. Getting the Pharmaceutical Industry to Develop Antimicrobial Medications. Infection Disease Society of America News Letter. December 2003

5. Torres-Viera C, Thauvin-Eliopoulos C, Souli M, et al. Activities of Taurolidine In Vitro and experimental enterococcal endocarditis. Antimicrobial Agents and Chemotherapy, June 2000, p 1720-1724

6. Shah C, Mittelman M, Costerton J W, et al. Antimicrobial activity of a novel catheter lock solution. Antimicrobial Agents and Chemotherapy, June 2002, p. 1674-1679

7. Sodemann K, Polaschegg H D, Feldmer B. Two years experience with Dialock and CLS (a new antimicrobial lock solution) 2001; 19: 251-254

8. Quarello F, Forneris G. Prevention of catheter-related bloodstream infection using an antimicrobial lock. Blood Purf 2002; 20:87-92

9. Allon M Prophylaxis against Dialysis Catheter Related Bacteremia with a novel Antimicrobial Lock Solution. Clinical Infectious Diseases 2003; 36: 1539-44

10. Betjes M, van Agteren M. Prevention of dialysis catheter-related sepsis with a citrate-taurolidine-containing lock solution. Nephrol Dial Transplant (2004)

11. Personal observation

12. Methicillin-Resistant Staphylococcus aureus Infections in Correctional Facilities—Georgia, California, and Texas, 2001-2003. MMWR 52 (41): 992-996, 2003. Centers for Disease Control and Prevention, USA. Posted Oct. 19, 2003

13. Karande P, Jain A, Mitragotri S. Discovery of Transdermal penetration enhancers by high-throughput screening. Nature Biotechnology, Vol 22, 2, February 2004

14. Breaking the Skin Barrier. 7 Articles relating to methods and devices to deliver drugs transdermal and 2 editorials. Advanced Drug Delivery, Mar. 27, 2004 Issue

15. Injectable Polymeric Biomaterials. 14 Papers describe the design and synthesis of injectable systems including photopolymers, degradeable polymers, and hydrogels. Biomaterials 23 (2002)

16. Harris J M, Veronese Peptide and Protein pegylation II—clinical evaluation. Advanced Drug Delivery Reviews 55 (2003) 1259-1260

17. Resomer Polymers. http://www.boehringer-ingelheim.com/finechem/products/resomer/resomer.htm

18. Promaxx protein-matrix drug bioerodible drug microspheres. Manufactured by Epic Therapeutics (Subsidiary of Baxter) www.baxterdrugdelivery.com

19. Hu Z, Wang C, Nelson K, Eberhart R. Controlled Release from Composite Silicone/Hydrogel Membrane. ASAIO Journal 2000; 46: 431-434

20. Richards C, Hoffman K, Bernhard J, et al. Development and Characterization of an Infection Inhibiting Urinary catheter. ASAIO Journal 2003; 49: 449-453

21. Duimel-Peeters I G P, Houwing R H, Teunissen C P. A Systematic Review of the Efficacy of Topical Skin Application of Dimethyl Sulfoxide on Wound Healing and as an Anti-Inflammatory Drug. Wounds 15(11):361-370, 2003

22. Active Dressing could speed healing (report on University of Dundee regarding development of proteins which activate the cell repair of the wound). Reported Saturday, 1 Jul. 2000. BBC Medical news

23. Greer D, Andreadis S. A Novel Role of Fibrin in Epidermal Healing: Plasminogen-Mediated Migration and Selective Detachment of Differentiated Keratinocytes. J Investigative Dermatol 121:1210-1216, 2003

24. Simon J, Nicholson J. Conformable Urethral Plug. U.S. Pat. No. 5,090,424, Feb. 25, 1992

25. Polaschegg H D. Prevention of Indwelling Device Related Infections: Composition and Methods. United States Patent Application, filed Feb. 3, 2004

26. Ledson M, Gallagher M, Robinson M et al. A Randomized Double-Blinded Placebo-Controlled Crossover Trial of Nebulized Taurolidine in Adult Cystic Fibrosis Patients Infected with Burkholderia cepacia. Journal of Aerosol Medicine, Vol 15, Ni, 2002, p 51-57

27. Willatts S M, Radford S, Leitermann M. Effect of the antiendotoxic agent, taurolidine, in the treatment of sepsis syndrome: a placebo-controlled, double-blind trial. Crit Care Med 1995;23:1033-39

28. Steinbach-Lebbin C, Ganz A J, Chang A, Waser P G. Über die Pharmakokinetik von Taurolin [Pharmacokinetics of Taurolin]. Arzneimittelforschung 1982;32:1542-6

29. http://hopkins-id.edu/idsa/hilies_bart_res.html

30. http://www.thedrugmonitor.com/vanco.html

31. http://www.rxkinetics.com/pktutorial/2_{—}5.html quoting Hyatt et al, Clin Pharmacokinet 28: 143, 1995

32. Blenkharn J I. In-vitro antibacterial activity of noxythiolin and taurolidine. J Pharm Pharmacol 1990;42:589-90

33. Reinke P. Plasmapheresis in the therapy of septic disease. Int J Artif Organs 1996;19:127-8. [Plasmafiltratrion]

34. Lonergan J M, Orlowski J P, Sato T, McHugh M J, Zborowski M. Extracorporeal Endotoxin Removal in a Canine Model of Septic Shock. ASAIO Journal 1994;40:M654-7 [Hemoperfusion]

35. Jaber B L, Pereira B J. Extracorporeal adsorbent-based strategies in sepsis. Am J Kidney Dis 1997;30:S44-56. [Hemoperfusion]

36. Rogiers P. Hemofiltration treatment for sepsis: is it time for controlled trials?. Kidney Int 1999;56Sup72:S99-103.) [Hemofiltration:]

37. Willatts S M, Radford S, Leitermann M. Effect of the antiendotoxic agent, taurolidine, in the treatment of sepsis syndrome: a placebo-controlled, double-blind trial. Crit Care Med 1995;23:1033-39

38. Kleophas W, Haastert B, Backus G, Hilgers P, Westhoff A, van Endert G. Long-term experience with an ultrapure individual dialysis fluid with a batch type machine. Nephrol Dial Transplant 1998;13:3118-25

39. Stange J, Mitzner S, Ramlow W, Gliesche T, Hickstein H, Schmidt R. A new procedure for the removal of protein bound drugs and toxins. ASAIO J 1993;39:M621-5

40. Drusano G., Preston S, Fowler C, et al. Relationship between Fluoroquinolone Area under the Curve: Minimum Inhibitory Concentration Ratio and the probability of Eradication of the Infecting Pathogen, in Patients with Nosocomial Pneumonia. The Journal of Infectious Diseases 2004; 189:1590-7

### U.S. Patent References

1. Patent Application EP03002292.5—Inventor: Polaschegg, Hans-Dietrich. Prevention of Indwelling Devices Related Infection: Composition and Methods

2. Graham et al: Controlled Release Device Chung, et al: Biodegradable sustained release preparation for treating periodontitus. U.S. Pat. No. 4,814,182

3. Gillis Therapeutic treatments using the direct application of antimicrobial metal compositions. Application Pub. No. US 2003/0086977

TABLE 1
Taurolidine Characteristics Considered Clinically beneficial^{1,2,5-12}

| ATTRIBUTES | DESCRIPTION |
|---|---|
| 1. No Bacterial Resistance | 25 years of clinical use and many in-vitro tests without |
| | any indication of bacterial resistance emerging |
| 2. Broad spectrum | Effective against substantially all clinically significant |
| antimicrobial effect | bacterial strains and some important fungi in published |
| | test results |
| 3. Excellent biocompatibility | Hi safety at concentrations considerably higher than |
| | the MIC (mean inhibitory concentration) and in direct |
| | contact to tissue and blood. Taurolidine breaks down |
| | in vivo to biologically safe materials |
| 4. Anti-inflammatory action | Reduced inflammatory response from infection, |
| | bactericidal treatments and tissue trauma |
| 4. Inactivates endotoxins | Taurolidine has been found to inactivate endotoxins |
| 5. Anti-sticking action & | Taurolidine treated surfaces resist attachment of blood |
| improved biocompatibility | components and bacteria and the certain synthetic |
| | materials exhibit improved biocompatibility |
| 6. High diffusibility through | Taurolidine is a small molecule which diffuses through |
| tissue and selected | human tissue and through some polymers |
| elastomers | |
| 7. Compatible with many | Taurolidine can be combined with other selected |
| antibiotics and drugs | chemical agents to enhance its therapeutic and |
| | delivery performance |
| 8. Near prevention of any | 2 controlled studies resulted in approximately 95% |
| infection from HD | reduction in infection compared to conventional heparin |
| catheters | lock |
| 9. Stable material | Taurolidine in powder or aqueous solution is stable |
| | over several years at room temperature. |

TABLE 2
Taurolidine Characteristics Considered Inhibiting to Usefulness^{1,2,5-12}

| ATTRIBUTES | DESCRIPTION |
|---|---|
| Requires an aqueous medium | Water is the preferred solvent to achieve action |
| 1. Strength in vials is very low | Saturation concentration is approximately 1% (i.e., 10 mg/ml) |
| | at room temperature in water. |
| 2. Minimum bactericidal | Taurolidine has an approximate 1000 times higher |
| concentration (MBC) is very | MBC than antibiotics. (i.e., MBC of 2 mg/ml vs. 2 ug/ml |
| high | for typical bacteria) |
| 3. Rapid body clearance | Taurolidine is quickly distributed to total body water |
| | and cleared from the body with a half life of |
| | approximately 70 minutes which is faster than for |
| | typical antibiotics. Oral and vaginal cavity topical use |
| | results in quick uptake by the local fluids and tissue |
| | and dislodgement from the space. |
| 4. Bactericidal action requires | Planktonic form of bacteria requires approximately 2 |
| substantial time | hours exposure to achieve killing action which is higher |
| | than typical antibiotics or other anti-infective agents. |
| | Bacterial death in a biofilm colony requires an even |
| | longer time. |
| 5. Painful to use | Direct contact with most tissue causes intense pain |

TABLE 3
Pharmacokinetic and Pharmacodynamic Values for central blood
values after a single IV dose of Vancomycin and Taurolidine.

| | Taurolidine | Vancomycin |
|---|---|---|
| Max available | 2% | 95% |
| Concentration in drug (Cᵥ) | (20 g/liter) | (10 g unit dose Pack) |
| Max Tolerated Dose | >30 g/day²⁷ | >5 g/day(See footnote a) |
| MIC (Minimum Inhibitory | ~500 μg/mL³² | <4 μg/mL²⁹ |
| Concentration) | | |
| T_{1/2} (Drug Half Life in | ~70/140 Minutes²⁸ | ~7 hours³⁰ |
| Blood) | (see Footnote b) | |
| Max Potential Delivery | ~60,000 mg/day | ~5 g/day |
| | (Limited by Fluid volume | (Limited by toxicity or |
| | patient can tolerate) | complications) |

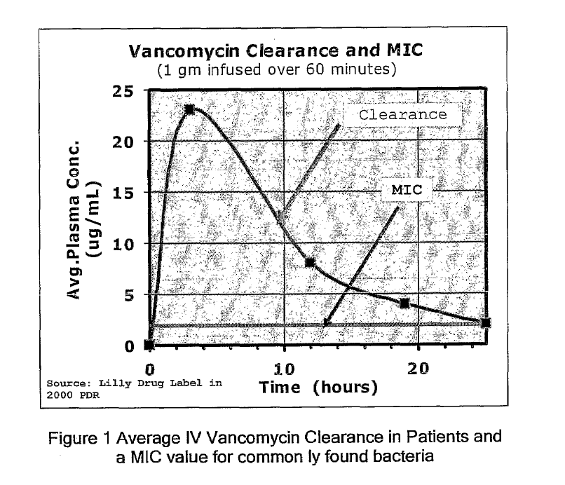

Surrogate Markers

| | | |
|---|---|---|
| Peak Plasma | 150 μg/ml²⁸ | 50 μg/mL |
| Concentration (C_{P}) | (Not applicable for Taurolidine) | |
| C_{P}/MIC | 0.3 | 12 |
| AUC/MIC | 2.26 | 125³¹ |
| (see Footnote c) | (see FIG. 4) | (see FIG. 1) |
| Σ Time > MIC (Amount of | Never | 24 hr |
| time over MIC) | (0%) | (100%) |

Notes:
a) Peak conc. Is 50 ug/ml, distribution vol. is 70 kg (70% of body wt.) with functional kidney and doses per day (ie, 5 × 10⁻⁵ × 0.7 × 10⁴ × 2 == 5 gm
b) PK of Taurolidine obtained from Ref 28 states Taurolidine rapidly equilibrates with body water in 30 minutes and is cleared with a first time constant of 0.3 which corresponds to a half life of ~2.3 hours This results in a time const of 0.57 and a half life of 70 minutes
c) Area Under the Curve (AUC) is determined from the clearance curves in FIG. 4, FIG. 1, and Referenc 31

What is claimed is:

1. A method for treating a localized bacterial infection, comprising locally applying Taurolidine to the bacterial infection.

2. A method according to claim 1, wherein the localized bacterial infection comprises one from the group consisting of: eye infections, ear infections, periodontal disease, tonsillitis, lung infections, urinary tract infections, vaginal infections, rectal infections, skin infections, wound infections, and surgical infections.

3. A method according to claim 1, wherein locally applying the Taurolidine comprises positioning the Taurolidine in a confined space within the body.

4. A method according to claim 3, wherein the confined space is naturally occurring.

5. A method according to claim 4, wherein the confined space comprises one from the group consisting of: an eye cavity, an ear cavity, the mouth, an oral cavity, a lung, the vagina, the rectum, an epidural/intrathecal space, and a urological space.

6. A method according to claim 3, wherein the confined space is artificially created.

7. A method according to claim 6, wherein the confined space comprises a pocket formed in tissue.

8. A method according to claim 6, wherein the confined space comprises a surgical pocket.

9. A method according to claim 6, wherein the confined space comprises a wound pocket.

10. A device for treating a localized bacterial infection, wherein the device carries Taurolidine therewith.

11. A device according to claim 10, wherein the device comprises one from the group consisting of: a bandage, an insert, a gel, a microsphere, and an injectable polymer.

12. A device according to claim 11, wherein the device comprises at least one selected from the group consisting of: polyglycol and polyethyleneglycol.

13. A device according to claim 10, wherein the device carries a reservoir of Taurolidine.

14. A device according to claim 13, wherein the device comprises a permeable membrane separating the reservoir from tissue.

15. A device according to claim 10, wherein the device comprises a material impregnated with Taurolidine.

16. A device according to claim 15 wherein the material comprises a polymer.

17. A device according to claim 15 wherein the material comprises an elastomer.

18. A device according to claim 15 wherein the material comprises silicone rubber.

19. A device for insertion into the body, the device comprising Taurolidine, whereby to render the device infection resistant.

20. A device according to claim 19, wherein the device comprises one from the group consisting of: a contact lens, ophthalmic fluid, tympanic tubes for ear drainage, periodontal insert, periodontal treatment tray, enteral feeding tubes, endotracheal tubes, urinary catheters, female urethra plugs, intra-uterine devices (IUD), hemodialysis catheters, blood catheters, intra-spinal catheters, intra-ventricular catheters, drainage tubes, active wound healing dressings, suture materials, adhesives, implantable ports and pumps, pacemakers, endoscopes, other insertable devices, breast implants, orthopedic devices with port, an injectable polymer, and bone cement.

21. A method for treating a lung infection, comprising inhaling a Taurolidine-carrying aerosol.

22. A method according to claim 21, wherein inhalation occurs through forced ventilation.

23. A method according to claim 21, wherein the Taurolidine particles are inhaled using a nebulizer.

24. A method for treating a lung infection, comprising inhaling Taurolidine particles.

25. A method according to claim 24, wherein inhalation occurs through forced ventilation.

26. A method according to claim 24, wherein the Taurolidine particles are inhaled using a dry powder inhaler.

27. A medication for treating bacterial infections, comprising Taurolidine carried by one from the group consisting of: gels, liquids, thixotropic gels, colloidal mixtures, dispersal suspensions, and injectable polymers, the medication having a Taurolidine concentration sufficiently high, and capable of being applied to a specific region for a sufficient period of time, to treat the infection.

28. A medication for treating bacterial infections, comprising Taurolidine carried by a microparticle, wherein the microparticle comprises one from the group consisting of: Resomer bioerodible plastics, Resomer bioerodible gels, microspheres, protein solid microspheres, colloidal materials, liposomes and composite silicone/hydrogel materials.

29. A method for using a reusable medical device, comprising positioning the reusable medical devices in a Taurolidine solution before use.

30. A composition comprising Taurolidine and various pluronic formulations.

31. A composition comprising Taurolidine and Hyaluronic acid (HA) and water.

32. A composition comprising Taurolidine and chitin and water.

33. A composition comprising Taurolidine, and chitosan or alginate, and water.

34. A composition comprising Taurolidine and cyclodextrin and water.

35. A composition comprising polyethylene glycol (PEG) based hydrogel system.

36. A composition comprising Taurolidine, wherein the composition is injectable and comprises a scaffold upon subcutaneous administration so as to provide a filler for a body cavity or act as a compliant implant and release the Taurolidine in vivo.

37. An endotracheal tube comprising:
a tube;
an inflatable collar attached to the outer wall of the tube, the inflatable collar comprising a material permeable to Taurolidine; and
a passageway extending along the tube and connected to the inflatable collar;
whereby Taurolidine may be introduced to the inflatable collar from outside the body in a flow and pressure controlled manner so as to inflate the collar and deliver Taurolidine around the periphery of the collar and/or the tube outer surface distal to the collar.

38. A method for ventilating a patient, comprising:
providing an endotracheal tube comprising:
a tube;
an inflatable collar attached to the outer wall of the tube, the outer inflatable collar comprising a material permeable to Taurolidine; and
a passageway extending along said tube and connected to the inflatable collar;
positioning the tube in the trachea;
introducing Taurolidine into the inflatable collar from outside the body in a flow and pressure controlled manner so as to inflate the collar and deliver Taurolidine around the periphery of the collar and/or the tube outer surface distal to the collar; and
ventilating the patient.

39. A urinary catheter comprising:
a tube;
an inflatable collar attached to the outer wall of the tube, the outer inflatable collar comprising a material permeable to Taurolidine; and
a passageway extending along said tube and connected to the inflatable collar;
whereby Taurolidine may be introduced to the inflatable collar from outside the body in a flow and pressure controlled manner so as to inflate the collar and deliver Taurolidine around the periphery of the collar and/or the catheter outer surface distal to the collar.

40. A method for treating a patient, comprising:
providing a urinary catheter comprising:
a tube;
an inflatable collar attached to the outer wall of the tube, the outer inflatable collar comprising a material permeable to Taurolidine; and
a passageway extending along said tube and connected to the inflatable collar;
whereby Taurolidine may be introduced to the inflatable collar so as to inflate the collar and deploy Taurolidine around the periphery of the collar;
positioning the tube in the urethra;
introducing Taurolidine into the inflatable collar from outside the body in a flow and pressure controlled manner so as to inflate the collar and deliver Taurolidine around the periphery of the collar and/or the catheter outer surface distal to the collar.

41. A Taurolidine depot adapted to be deposited within the body during a medical procedure, and thereafter deliver Taurolidine to the tissue adjacent to the body.

42. A Taurolidine depot according to claim 41, wherein the Taurolidine depot has a cylindrical shape.

43. A method for treating a patient, comprising:
providing a Taurolidine depot adapted to be deposited within the body during a medical procedure, and thereafter deliver Taurolidine to the tissue adjacent to the body; and
depositing the Taurolidine depot within the body during a medical procedure.

44. A method for treating blood, comprising:
removing blood from the body;
treating the removed blood with Taurolidine; and
returning the treated blood to the body.

45. Apparatus for treating blood, comprising:
removal apparatus for removing blood from the body;
treatment apparatus for treating the removed blood with Taurolidine; and
return apparatus for returning the treated blood to the body.

46. Apparatus according to claim 45, wherein the treatment apparatus comprises a dialysis type machine.

47. Apparatus according to claim 45, wherein the treatment apparatus comprises a cartridge for exposing the removed blood to a supply of Taurolidine.
